(19) United States
(12) Patent Application Publication
(10) Pub. No.: US 2016/0074022 A1
(43) Pub. Date: Mar. 17, 2016

(54) Separable Specimen Collection Device

(71) Applicant: Hologic, Inc., Bedford, MA (US)

(72) Inventors: Ryan Oliva, Marlborough, MA (US); Steven Hecht, Boston, MA (US); Howard B. Kaufman, Newton, MA (US)

(21) Appl. No.: 14/856,604

(22) Filed: Sep. 17, 2015

Related U.S. Application Data
(60) Provisional application No. 62/051,675, filed on Sep. 17, 2014.

(51) Int. Cl. A61B 10/02; A61B 10/00

(52) U.S. Cl. CPC A61B 10/0291; A61B 10/0096; A61B 2010/0216

(57) ABSTRACT

The invention provides a device, system and method for sample collection wherein a sample can be separated into multiple portions of sample at the site of collection. The separation of portions of sample occurs by separating parts of the specimen collection device. Each of the separable parts contacting a portion of sample can be deposited into separate vessels. The device reduces the risk of sample contamination, because the separable parts can be separated by the action of a release mechanism that allows a user to separate the parts without touching or otherwise contacting the sample collecting region. The release mechanism may be trigger-activated to expel a separable part of the device.

### CROSS-REFERENCE TO RELATED APPLICATION

This application claims the benefit of and priority to U.S. Provisional Application Ser. No. 62/051,675, filed Sep. 17, 2014, the contents of which are incorporated by reference herein in their entirety.

### FIELD OF THE INVENTION

The present invention relates to sample collection devices, particularly devices capable of separating a sample into subparts.

### BACKGROUND

The Papanicolaou (Pap) test is a widely-used method of cervical screening that detects abnormalities in cervical and endometrial cells, including pre-cancerous and cancerous lesions. The Pap test is widely used because it is simple, minimally invasive, and inexpensive. The test generally involves taking a sample of cells from the cervix using a collection device, and performing cytological analysis of cells for diagnostic characteristics that are indicative of the presence of disease. Early detection of cervical abnormalities is essential for effective treatment, and regular Pap screening has reduced the number of annual deaths in the United States due to cervical cancer by more than 60% since its introduction in 1955 (National Cancer Institute).

To collect a cervical sample, clinicians use a variety of devices including swabs, spatulas, and brushes. In some instances, it may be desirable to collect samples from inside the cervix (endocervical canal) and from the surface of the cervix (outer cervix). A common method involves scraping the outer opening of the cervix with a spatula and then using a separate endocervical brush to collect cells from the central opening of the cervix and the endocervical canal. The collection devices are submerged in a vial containing liquid medium and stirred to release cells into the medium. Such sampling may be done with separate devices or with a device having multiple components, e.g., as disclosed in U.S. Pat. No. 8,152,739, incorporated herein by reference in its entirety. Separable systems, e.g., as shown in U.S. Pat. No. 8,152,739, often require excessive manipulation to separate the multiple components, presenting a risk of contamination to an operator or introduction of foreign material into the sample.

Conventional methods require fixation of a portion of the collected cells on a slide for evaluation of cell morphology. The slides may be prepared by hand (“Pap Smear”), however superior results can be obtained with automated systems, such as the ThinPrep® Pap test combined with the ThinPrep® Imaging System, available from Hologic, Inc. (Bedford, Mass.). This methodology is superior to the conventional Pap smear because of improved accuracy and increased disease detection (citation—Surveillance, Epidemiology, and End Results (SEER) Program. SEER Database: Incidence—SEER 9 Regs Public-Use, November 2004 Sub (1973-2002), National Cancer Institute, DCCPS, Surveillance Research Program, Cancer Statistics Branch, released April 2005, based on November 2004 submission). Once the cells are fixed, the sample may be screened for atypical cells and other cytologic abnormalities.

Recent advances in genetic screening technologies have made it possible to screen for genetic changes indicative of cancer or infection. For example, cervical samples may be collected and screened for molecular diagnostics using a genetic assay, such as hybrid assay, multiplex PCR, or direct sequencing. The sample may be screened against a database of genetic markers to identify a woman's risk of cervical cancer, by typing for HPV-16, HPV-18, HPV-31, HPV-33, HPV-35, HPV-39, HPV-45, HPV-51, HPV-52, HPV-56, HPV-58, HPV-68, HPV-73 or HPV-82. The screening may be based upon DNA, RNA, or some combination thereof. Commercial systems for diagnostic screening for HPV are available from Hologic, Inc., e.g., Cervista® HPV or APTIMA® HPV assays.

While the standard of care for cervical screening in the United States is the Pap test, the U.S. Food and Drug Administration recently cleared the way for genetic testing alone to be used to screen women for cervical cancer. However, groups such as the American Medical Women's Association have expressed concern that genetic testing, exclusive of morphology screening, will result in too many women receiving treatment when those women are merely carriers of HPV and don't have any immediate risk of developing cervical cancer. See “FDA approves Roche Genetic Test as an Alternative to Pap Smear for Cervical Cancer Screening,” Associated Press, Apr. 24, 2014, incorporated by reference herein in its entirety.

### SUMMARY

The present invention provides sample collection devices capable of separating a sample into subparts at the site of collection; as well as methods for collecting a sample with the sample collection device. In a preferred embodiment, the device comprises a brush with a sample collecting region that includes multiple separable parts, each of which can be deposited into separate vessels. The device reduces the risk of sample contamination because the separable parts can be separated by the action of a release mechanism that allows a user to separate the parts without touching or otherwise contacting the sample collecting region. The device additionally assures that two similar samples are compared, as opposed to two samples from different locations or of different quality. A preferred embodiment of the release mechanism is a trigger-activated plunger that expels a portion of the brush.

The device and method can be employed to collect a cervical sample during a cervical examination. The clinician can insert the distal end of the brush into the cervical opening and, using the handle, rotate the brush several times to collect a cervical sample, including cells from within and on the surface of the cervix. Once the brush is withdrawn, the clinician activates the release mechanism, thereby separating the brush into multiple parts, each part having a portion of the sample. Upon separating, the multiple parts are inserted directly into separate vessels that contain and store the divided portions of sample. In an embodiment, a first sample contained in a first vessel is used for cytology and a second sample contained in a second vessel is used for molecular diagnostics.

With this method, multiple vessels containing sample can be obtained from a single pass with the cervical brush, obviating the need to take multiple samples with multiple brushes, or taking aliquots of a single sample and thereby risking contamination. Samples collected in a single pass can be easily divided and deposited into separate vessels or different parts of the same vessel. This allows different tests to be performed easily. Another advantage of the disclosed method over sequential collection methods is that two samples obtained in a single collection are more congruent with each other than two samples collected independently. Thus, the invention is in contrast to sequential collection methods, whereby a first sample collection may pick up most of the cells of interest, leaving the second collection with an inferior sample.

### BRIEF DESCRIPTION OF THE DRAWINGS

FIG. 1A shows a specimen collection device suitable for sampling endocervical and ectocervical cells.

FIG. 1B shows a specimen collection device with a separable brush member separated from the brush head.

FIG. 1C shows a specimen collection device with a separable brush member separated from the brush head.

FIG. 2A shows a specimen collection device.

FIG. 2B shows a specimen collection device with a separable brush member separated from the brush head.

FIG. 2C shows a specimen collection device with a separable brush member separated from the brush head.

FIG. 3A shows a specimen collection device.

FIG. 3B shows a specimen collection device with a separable brush member with a foundation and a shaft, separated from the brush head.

FIG. 4A shows a specimen collection device.

FIG. 4B shows a specimen collection device with a separable brush member separated from the brush head by the action of a plunger.

FIG. 4C shows another view of a detachable brush member 140 uncoupled from the brush head 120.

FIG. 5 shows a specimen collection device with bristles.

FIG. 6 shows a specimen collection system including a specimen collection device of the invention and vessels for collecting the specimen.

FIG. 7 shows a specimen collection system including a vessel including two separate compartments.

### DETAILED DESCRIPTION

The present disclosure describes a specimen collection device configured for separating a sample at the site of collection, and methods of using the specimen collection device. The sample can be partitioned by separating the specimen collection device into more than one part, each part capable of carrying a portion of the sample. The separable parts are designed to be decoupled using a release mechanism that obviates the need for the user to touch or otherwise contact the sample collecting regions of the specimen collection device. This hands-free release mechanism reduces the likelihood of contamination of the sample. Unlike conventional methods, whereby a clinician takes multiple samples using multiple brushes, the invention results in specimen collection that is cheaper, less time-consuming, and less invasive to the patient. Furthermore, because multiple passes with cervical brushes can lead to irritation and bleeding, overall patient satisfaction is improved. Also, the disclosed method provides a single, more homogenous and representative sample over sequential sequencing.

Also described herein is a specimen collection system that employs the specimen collection device to collect a sample and deposit the sample into one or more vessels. This system provides advantages over conventional technologies, wherein a single sample is taken and then that sample is partitioned before the sample is assayed. These state-of-the-art methods often require a vessel, containing the sample, to be transported to a laboratory where multiple aliquots are removed for sampling. Different specimen collection media are appropriate for different types of tests that can be done, such as PreservCyt® Solution for cytology and APTIMA® STM for RNA analysis. With each sample removal, the vessel must be reopened, the sample divided and the vessel reclosed each time. This multi-step process introduces transport and logistical complications and also increases the likelihood of unwanted contamination of samples.

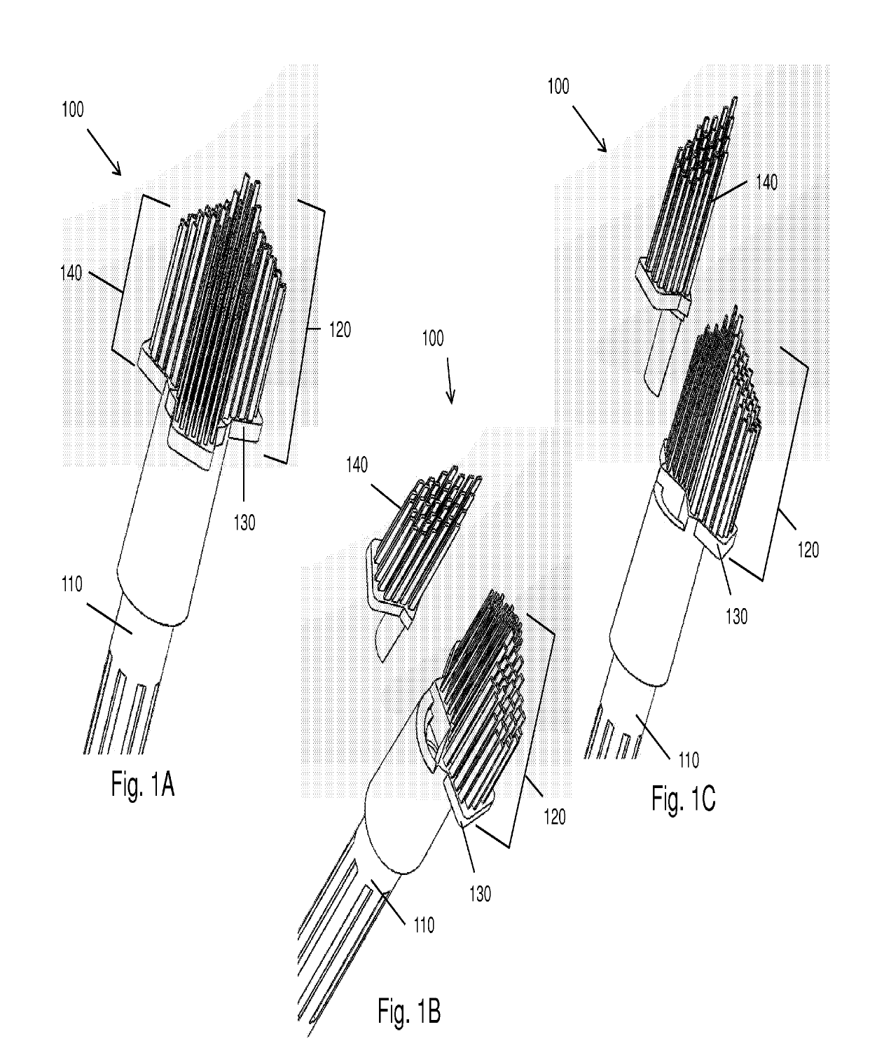

FIG. 1A shows a specimen collection device 100 suitable for use with the methods described herein. The specimen collection device 100 includes a handle 110 having a proximal and distal end. The distal end of the handle 110 is coupled to a base 130 of a brush head 120. The base 130 can be coupled to the handle 110 in a substantially perpendicular arrangement. A detachable brush member 140 is coupled to the brush head 120. FIGS. 1B and 1C show two views of a detachable brush member 140 uncoupled from a brush head 120.

Neither the shape of the brush head 120 nor the shape of the detachable brush member 140 is limited to the shapes depicted in FIGS. 1B and 1C. Subsequent figures show other nonlimiting shapes of the brush head 120 and the detachable brush member 140. The brush head 120 and the detachable brush member 140 can be oblong, conical, trapezoidal, fan-shaped, round, pointed or square. The shape of the detachable brush member 140 and the shape of the brush head 120 can be the same or different from one another.

The detachable brush member 140 and the brush head 120 can be coupled to each other with a variety of mechanisms, including snaps, tabs, perforations, pressure fittings, magnets, retaining rings or adhesives. The detachable brush member 140 can be molded to the brush head 120.

The detachable brush member 140 can be decoupled from the brush head 120 by means of a trigger mechanism (not pictured) that allows a user to separate the brush parts without touching or otherwise contacting the brush head 120 or the detachable brush member 140. The trigger mechanism may comprise a spring, a hook, a latch, a magnet, a holding ring, other apparatuses known in the art or any combination thereof. The trigger mechanism may be made of plastic, metal or another resilient material known in the art.

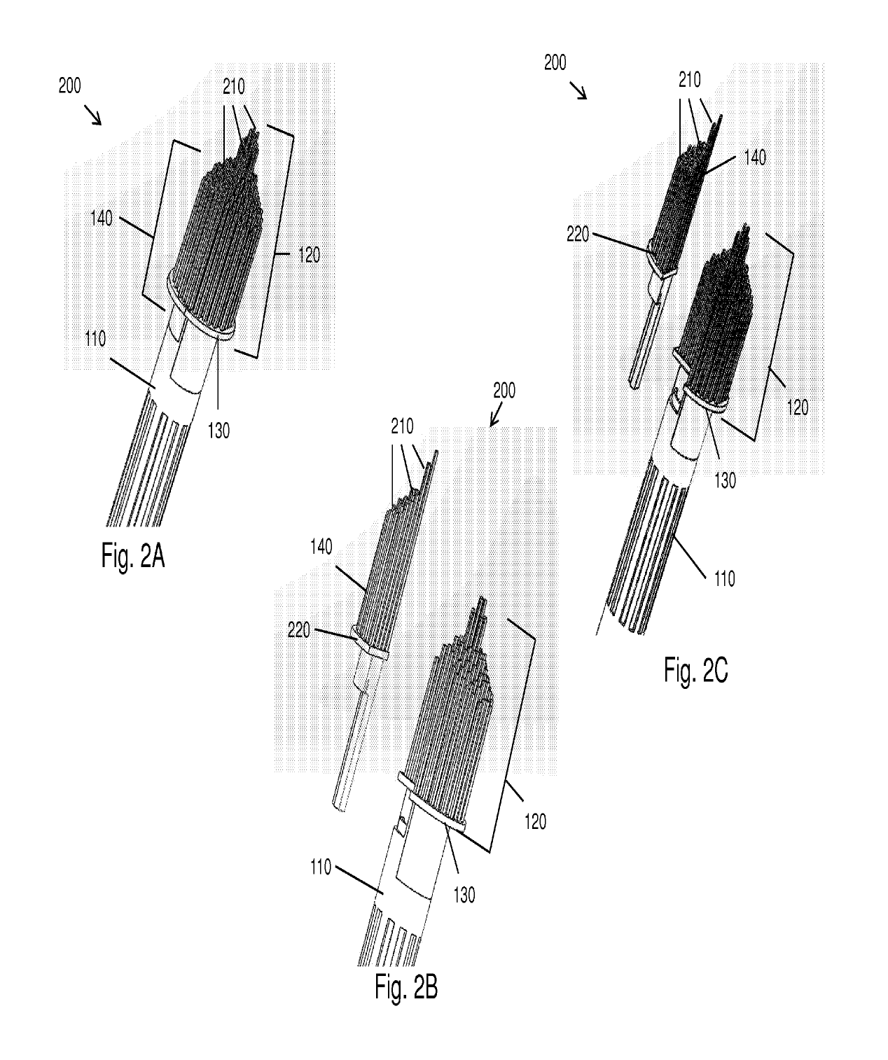

FIG. 2A shows a specimen collection device 200 suitable for use with the methods described herein. The specimen collection device 200 includes a handle 110 having a proximal and distal end. The distal end of the handle 110 is coupled to a base 130 of a brush head 120. A detachable brush member 140 is coupled to the brush head 120. The brush head 120 includes protrusions 210 for collecting cellular material. FIG. 2B shows an example of the detachable brush member 140 uncoupled from the brush head 120. The detachable brush member 140 comprises protrusions 210 for collecting cellular material and a foundation 220. FIG. 2C shows another view of the detachable brush member 140 uncoupled from the brush head 120. The detachable brush member 140 comprises protrusions 210 for collecting cellular material and a foundation 220.

The protrusions 210 can be bristles, rods, fibers, swabs or bumps. The protrusions 210 can be rigid or flexible. The protrusions 210 can be made of plastic, nylon, rubber, metal, wood or a medical-grade polymer material. Other materials known to those skilled in the art may also be used to create protrusions suitable for particular applications.

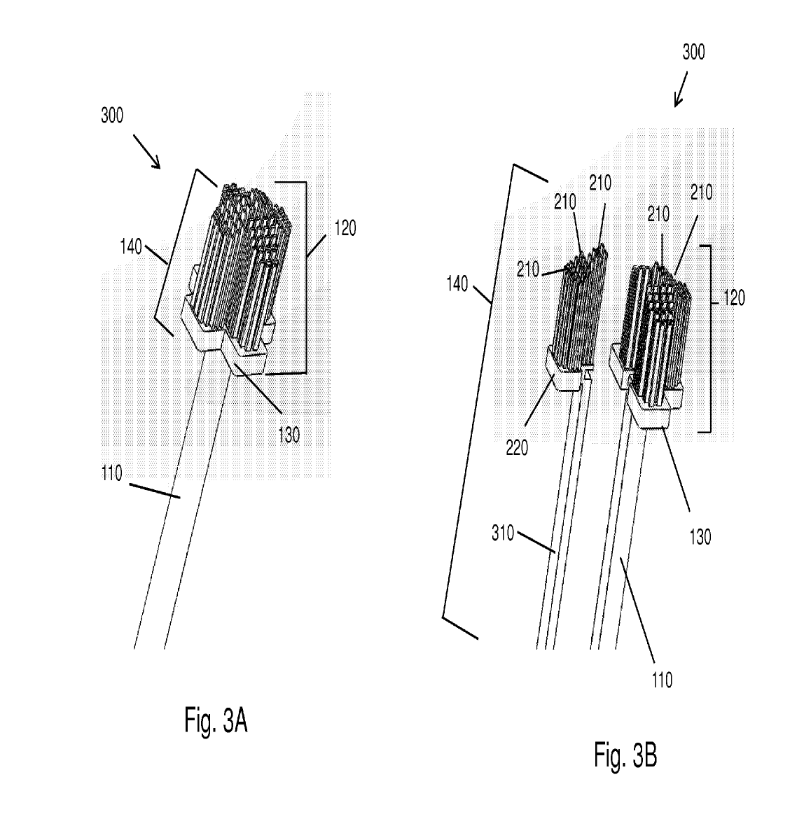

FIG. 3A shows a specimen collection device 300 suitable for use with the methods described herein. The specimen collection device 300 includes a handle 110 having a proximal and distal end. The distal end of the handle 110 is coupled to a base 130 of a brush head 120. A detachable brush member 140 is coupled to the brush head 120. FIG. 3B shows an example of the detachable brush member 140 uncoupled from the brush head 120. The detachable brush member 140 comprises a foundation 220 coupled to a shaft 310 and protrusions 210 for collecting cellular material. The brush head 120 includes protrusions 210 for collecting cellular material. The protrusions 210 can be bristles, rods, fibers, swabs or bumps. The protrusions 210 can be rigid or flexible. The protrusions 210 can be made of plastic, nylon, rubber, metal, wood or a medical-grade polymer material. Other materials known to those skilled in the art may also be used to create protrusions suitable for particular applications.

The handle 110 and the shaft 310 can be coupled to each other by a variety of arrangements, including but not limited to the following: the shaft 310 nested within the handle 110; the shaft 310 adhered to the handle 110 with for example, a biocompatible glue; the shaft 310 molded to the handle 110; or the shaft 310 and handle 110 interlocked together. The shaft 310 and handle 110 can also be coupled using a ring, a clamp, a latch, a pressure fitting or the like.

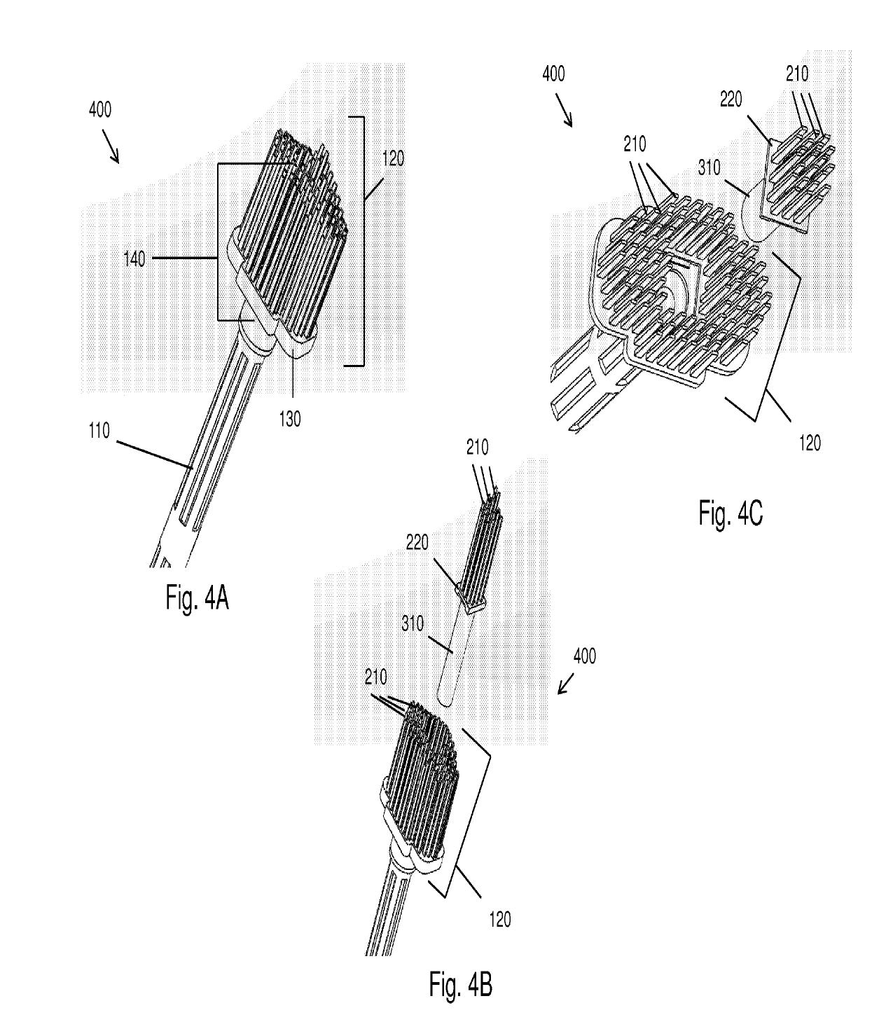

FIG. 4A shows a specimen collection device 400 suitable for use with the methods described herein. The specimen collection device 400 includes a handle 110 having a proximal and distal end. The distal end of the handle 110 is coupled to a base 130 of a brush head 120. A detachable brush member 140 is coupled to the brush head 120. In the nonlimiting embodiment of FIG. 4A, the detachable brush member 140 includes a shaft (not pictured) nested within the handle 110. FIG. 4B shows an example of the detachable brush member 140 uncoupled from the brush head 120. The brush head 120 includes protrusions 210 for collecting cellular material. The detachable brush member 140 comprises a foundation 220 coupled to a shaft 310 and protrusions 210 for collecting cellular material. The protrusions 210 can be bristles, rods, fibers, swabs or bumps. The protrusions 210 can be rigid or flexible. The protrusions 210 can be made of plastic, nylon, rubber, metal, wood or a medical-grade polymer material. Other materials known to those skilled in the art may also be used to create protrusions suitable for particular applications.

The detachable brush member 140 can be uncoupled from the brush head 120 by the action of a plunger (not pictured) capable of expelling the shaft 310 from within the handle 110. The plunger can be activated manually by a user by pressing a button, activating a spring mechanism or sliding a rail.

FIG. 4C shows another view of a detachable brush member 140 uncoupled from the brush head 120. The brush head 120 includes protrusions 210 for collecting cellular material. The detachable brush member 140 comprises a foundation 220 coupled to a shaft 310 and protrusions 210 for collecting cellular material. The protrusions 210 can be bristles, rods, fibers, swabs or bumps. The protrusions 210 can be rigid or flexible. The protrusions 210 can be made of plastic, nylon, rubber, metal, wood or a medical-grade polymer material. Other materials known to those skilled in the art may also be used to create protrusions suitable for particular applications.

The detachable brush member 140 can be uncoupled from the brush head 120 by the action of a plunger (not pictured) capable of expelling the shaft 310 from within the handle 110. The plunger can be activated manually by a user by pressing a button, activating a spring mechanism or sliding a rail.

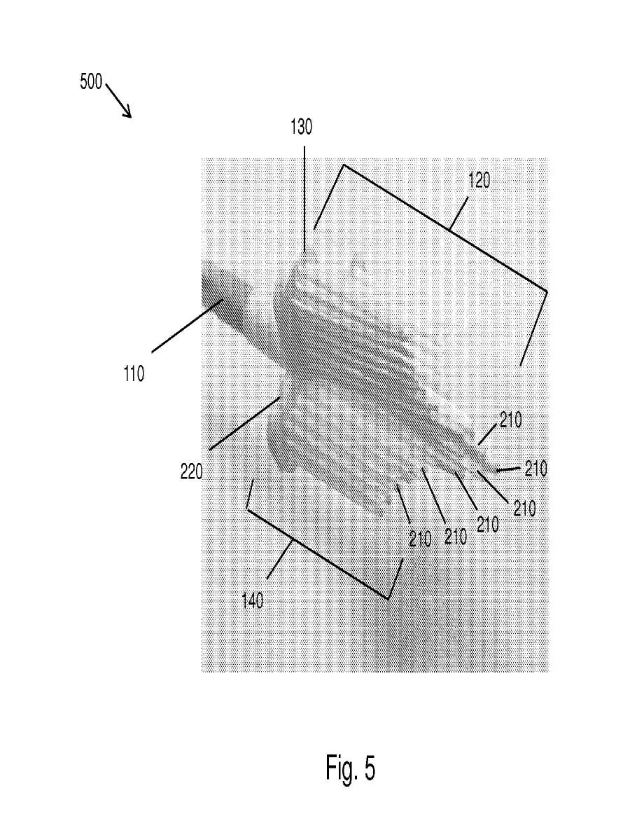

FIG. 5 shows an embodiment of the specimen collection device 500 suitable for use with the methods described herein. The specimen collection device 500 includes a handle 110 having a proximal and distal end. The distal end of the handle 110 is coupled to a base 130 of a brush head 120. A detachable brush member 140 is coupled to the brush head 120. The brush head 120 includes protrusions 210 for collecting cellular material. The detachable brush member 140 includes protrusions 210 for collecting cellular material. The protrusions 210 are in the form of soft bristles, but the protrusions 210 can be bristles, rods, fibers, swabs or bumps. The protrusions 210 can be rigid or flexible. The protrusions 210 can be made of plastic, nylon, rubber, metal, wood or a medical-grade polymer material. Other materials known to those skilled in the art may also be used to create protrusions suitable for particular applications.

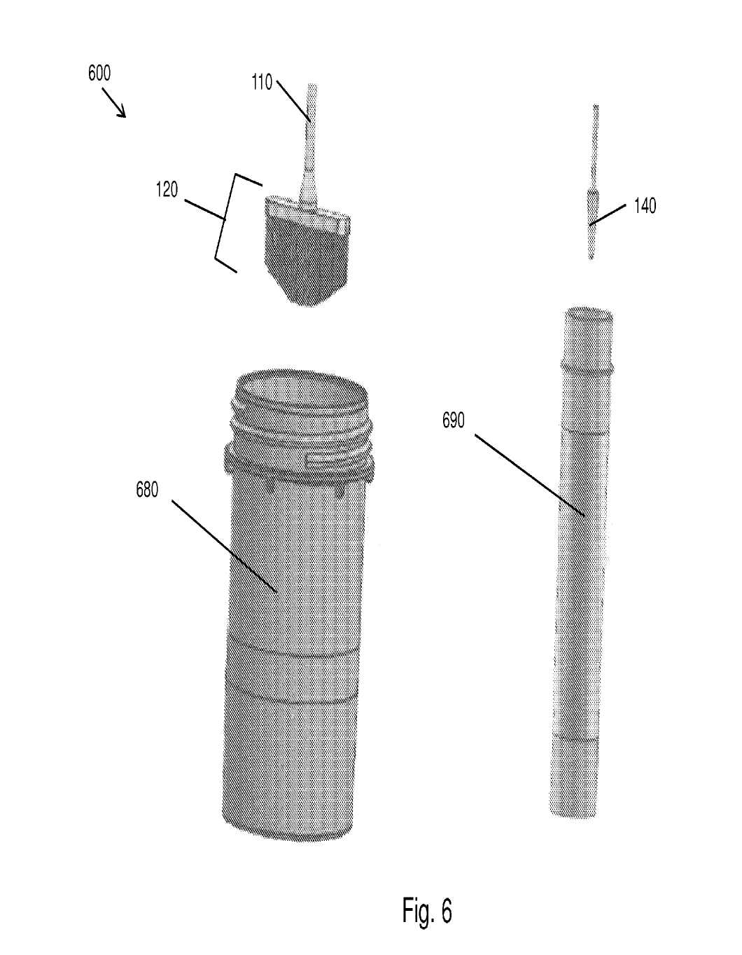

FIG. 6 shows a specimen collection system 600 suitable for use with the methods described herein. The specimen collection system 600 includes a specimen collection device (not pictured intact) including a handle 110, a brush head 120 and detachable brush member 140. The specimen collection system 600 includes a first vessel 680 and a second vessel 690. The first vessel 680 is suitable for holding a first cellular sample. The second vessel 690 is suitable for holding a second cellular sample. The first vessel and the second vessel can each comprise a detergent, an alcohol, a buffer or the like. The detergent can be Tween-20, Triton X-100 or any other detergent known in the art. The alcohol can be methanol, ethanol, isopropanol or any other alcohol known in the art. The buffer can be Tris, PBS or any other buffer known in the art.

The first cellular sample can be obtained with the brush head 120. Alternatively the first cellular sample can be obtained with the detachable brush member 140. The second cellular sample can be obtained with the brush head 120. Alternatively the second cellular sample can be obtained with the detachable brush member 140.

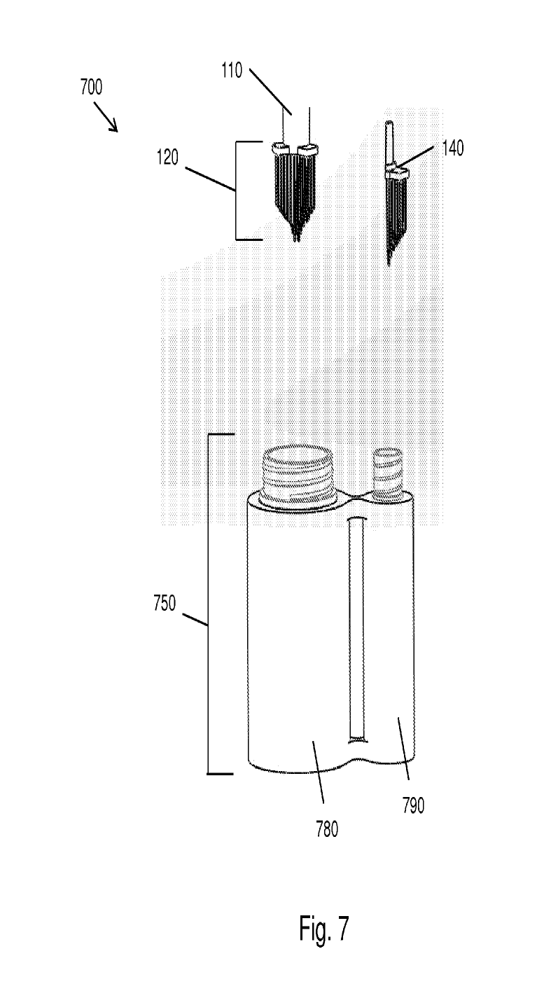

FIG. 7 shows a specimen collection system 700 suitable for use with the methods described herein. The specimen collection system 700 includes a specimen collection device (pictured as pieces) including a handle 110, a brush head 120 and detachable brush member 140. The specimen collection system 700 includes a vessel 750 comprising a first compartment 780 and a second compartment 790. The first compartment 780 is suitable for holding a first cellular sample. The second compartment 790 is suitable for holding a second cellular sample. The first compartment and the second compartment can each comprise a detergent, an alcohol, a buffer or the like. The detergent can be Tween-20, Triton X-100 or any other detergent known in the art. The alcohol can be methanol, ethanol, isopropanol or any other alcohol known in the art. The buffer can be Tris, PBS or any other buffer known in the art.

The first cellular sample can be obtained with the brush head 120. Alternatively the first cellular sample can be obtained with the detachable brush member 140. The second cellular sample can be obtained with the brush head 120. Alternatively the second cellular sample can be obtained with the detachable brush member 140.

The specimen collection devices shown in FIGS. 1A through 5 can be used in a method of specimen collection. The method comprises providing the specimen collection device and using it to collect a cellular sample. Once collected, the specimen collection device allows a sample to be easily partitioned. For example, one portion could be used to prepare a cytology slide to examine cell morphology while another portion can be used for genetic screening for HPV markers. The genetic screening may include any known method for genetic screening such as hybrid assay, real-time PCR, digital PCR, next-generation sequencing, Sanger sequencing, mass spectrometry, etc.

The specimen collection devices of the invention can also be used for collecting other cellular samples such as an oral sample, a buccal sample, a rectal sample, a nasal sample and the like. The cellular sample can then be assayed using a diagnostic system such as the ThinPrep® Pap test combined with the ThinPrep® Imaging System (Hologic, Inc.), the SurePath™ system (Becton Dickinson), or other diagnostic systems known in the art.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A specimen collection device, comprising:
a handle having a proximal and a distal end;
a brush head, comprising a base, and being coupled to the distal end of the handle; and
a detachable brush member, configured to be uncoupled from the brush head.

2. The specimen collection device of claim 1, wherein the brush head comprises protrusions for collecting cellular material.

3. The specimen collection device of claim 1, wherein the detachable brush member comprises protrusions for collecting cellular material.

4. The specimen collection device of claim 2, wherein the cellular material comprises whole cells, partial cells, or nucleic acids.

5. The specimen collection device of claim 3, wherein the cellular material comprises whole cells, partial cells, or nucleic acids.

6. The specimen collection device of claim 1, wherein the detachable brush member comprises a foundation at a proximal end of the detachable brush member.

7. The specimen collection device of claim 6, wherein the foundation is coupled to a shaft.

8. The specimen collection device of claim 7, wherein the handle and shaft are coupled adjacent to each other.

9. The specimen collection device of claim 7, wherein the shaft is nested within the handle.

10. The specimen collection device of claim 1, wherein the detachable brush member is uncoupled from the brush head by a trigger mechanism.

11. A specimen collection system, comprising:
a specimen collection device having:
a handle having a proximal and a distal end,
a brush head, comprising a base, and being coupled to the distal end of the handle, and
a detachable brush member, configured to be uncoupled from the brush head; and
a first vessel for holding a first cellular sample obtained with the specimen collection device.

12. The system of claim 11, wherein the first cellular sample is obtained with the brush head.

13. The system of claim 11, wherein the first cellular sample is obtained with the detachable brush member.

14. The system of claim 11, further comprising a second vessel for holding a second cellular sample.

15. The system of claim 14, wherein the first cellular sample is obtained with the brush head and the second cellular sample is obtained with the detachable brush member.

16. The system of claim 14, wherein the first vessel comprises an alcohol.

17. The system of claim 14, wherein the second vessel comprises a detergent.

18. The system of claim 16, wherein the second vessel comprises a detergent.

19. A method of obtaining a cellular sample, comprising:
providing a specimen collection device having a handle having a proximal and a distal end, a brush head, comprising a base, and being coupled to the distal end of the handle, and a detachable brush member, configured to be uncoupled from the brush head; and
collecting a first cellular sample with the specimen collection device.

20. The method of claim 19, further comprising delivering the first cellular sample to a first vessel.

21. The method of claim 20, further comprising collecting a second cellular sample with the specimen collection device.

22. The method of claim 21, further comprising delivering the second cellular sample to a second vessel.

23. The method of claim 22, wherein the first cellular sample is collected with the brush head and the second cellular sample is collected with the detachable brush member.
